(19) United States
(12) Patent Application Publication
(10) Pub. No.: US 2015/0010482 A1
(43) Pub. Date: Jan. 8, 2015

(54) FUNCTIONAL OIL POLYOL ACRYLIC GRAFT COPOLYMERS AND THEIR USE IN PERSONAL CARE APPLICATIONS

(71) Applicant: Akzo Nobel Chemicals International B.V., Amersfoort (NL)

(72) Inventors: Laurie Marshall, Center Valley, PA (US); Samuel Anthony Vona, JR., Highland, NY (US); Mojahedul Islam, White House Station, NJ (US); Jaime Dion Hamm, Hillsborough, NJ (US)

(21) Appl. No.: 14/366,354

(22) PCT Filed: Dec. 18, 2012
(86) PCT No.: PCT/EP2012/075876

§ 371 Date: Jun. 18, 2014

Related U.S. Application Data
(60) Provisional application No. 61/578,626, filed on Dec. 21, 2011.

(30) Foreign Application Priority Data
Mar. 14, 2012 (EP) 12159518.5

(51) Int. Cl. A61K 8/91; A01N 25/10; A61Q 19/00; A61Q 5/12; A61Q 17/04; A61K 47/32; A61Q 5/06

(52) U.S. Cl. CPC A61K 8/91; A61K 47/32; A01N 25/10; A61Q 5/06; A61Q 5/12; A61Q 17/04; A61Q 19/00; A61K 2800/40
USPC 424/59; 424/70.11; 514/772.1

(57) ABSTRACT

Personal care compositions include a hydroxyl functional vegetable oil polyol acrylic graft copolymer and a personal care active ingredient, and methods of use of a hydroxyl functional vegetable oil polyol acrylic graft copolymers in personal care applications. The hydroxyl functional vegetable oil polyol acrylic graft copolymer is obtained from a modified epoxidized vegetable oil polymerized in the presence of a ethylenically unsaturated monomer.

### FIELD OF THE INVENTION

The present invention relates to personal care compositions comprising hydroxyl functional oil polyol acrylic graft copolymers, and methods of use thereof in personal care applications.

### BACKGROUND OF THE INVENTION

Copolymers have been used in personal care formulations, for example as waterproofing agents for skin care applications or as styling agents for hair care applications. The conventional copolymers used in such personal care formulations, however, have predominantly been synthetic and/or are not easily biodegradable.

From the art, it is known that U.S. Pat. No. 5,728,796 discloses a process for reacting an epoxide-containing compound with an aliphatic hydroxyl containing compound in the presence of a sulfonic acid catalyst. This process is used to form epoxy resins having high molecular weights.

U.S. Pat. No. 4,012,559 discloses a radiation curable coating composition having an acrylic copolymer, a polyfunctional compound having a molecular weight not greater than 2,000, and an epoxidized vegetable oil. However, it is explained that the cured compositions are coated onto metal to form precoated metal products.

Guo, et al, Rigid Polyurethane Foams Based on Soybean Oil, Journal of Applied Polymer Science, Vol. 77, 467-473 (2000) teaches the reaction of an epoxidized soybean oil with methanol to form an epoxidized soybean oil polyol. However, it is described that is the epoxidized soybean oil polyol is used to prepare polyurethane foams for use in thermal insulation and packaging.

U.S. Patent Application Publication No. 2008/0302694 discloses a radiation curable coating composition having an epoxidized vegetable oil oligomer prepared from the reaction of an epoxidized vegetable oil and a hydroxyl functional acrylate or hydroxyl functional methacrylate in the presence of a sulfonic acid catalyst. However, this composition is described as used as a coating for packaging materials in food storage. U.S. Pat. No. 4,212,781 discloses processes for modifying an epoxy resin through a reaction with a copolymerizable monomer in the presence of an initiator. A graft polymer is formed from the epoxy resin by the grafting an addition polymer onto the aliphatic backbone of the epoxy resin. However, it is disclosed that the process is useful for making polymer blends for coating compositions.

Accordingly, there is a need to provide an alternative to conventional personal care polymers. More specifically, there is a need to provide naturally derived polymers that are readily biodegradable and sustainable that can be used in personal care formulations, and in particular those that provide waterproofing functionality in skin care formulations and/or styling functionality, including film formation and high humidity curl retention, in hair care formulations.

### SUMMARY OF THE INVENTION

In an aspect, the present invention is directed to a personal care composition comprising a hydroxyl functional vegetable oil polyol acrylic graft copolymer and a cosmetic or personal care active ingredient. The hydroxyl functional vegetable oil polyol acrylic graft copolymer may be prepared from a modified epoxidized vegetable oil that has been polymerized in the presence of at least one ethylenically unsaturated monomer.

### DETAILED DESCRIPTION OF THE INVENTION

The present invention generally is directed to a personal care composition comprising a hydroxyl functional vegetable oil polyol acrylic graft copolymer and a cosmetic or personal care active ingredient. The hydroxyl functional vegetable oil polyol acrylic graft copolymer may be prepared from a modified epoxidized vegetable oil that has been polymerized in the presence of a ethylenically unsaturated monomer. It has been found that the hydroxyl functional vegetable oil polyol acrylic graft copolymer has the capability of providing improved water and humidity resistance over conventional polymers typically used in hair care and skin care applications that are based solely on petroleum-based polymers. The inventive polymers offer improved biodegradability and more eco-friendly formulations that is of particular interest in personal care applications.

As used herein, the following terms generally have the meaning as indicated, but these meanings are not meant to limit the scope of the invention if the benefit of the invention is achieved by inferring a broader meaning to the following terms.

In some embodiments, the hydroxyl functional vegetable oil polyol acrylic graft copolymers are prepared by reacting an epoxidized vegetable oil with a hydroxyl functional material in the presence of an acid catalyst to form a hydroxyl functional oil polyol, and reacting the hydroxyl functional oil polyol with an ethylenically unsaturated monomer component in the presence of an initiator to form the hydroxyl functional oil polyol acrylic graft copolymer. The invention also includes personal care formulations utilizing the vegetable oil polyol acrylic graft copolymers as water and humidity resistant additives. One of the benefits associated with the vegetable oil polyol acrylic graft copolymers are their high level of biodegradability and eco-friendly (not petroleum-based) characteristics.

In some embodiments, the invention includes processes for producing a hydroxyl functional oil polyol acrylic graft copolymer. Such processes can be performed in a single reactor or in multiple reactors. In some embodiments of the invention, a hydroxyl functional oil polyol acrylic graft copolymer is prepared by a method comprising the steps of reacting an epoxidized vegetable oil with a hydroxyl functional material in the presence of an acid catalyst to form a hydroxyl functional oil polyol and reacting the hydroxyl functional oil polyol with an ethylenically unsaturated monomer component in the presence of an initiator to form the hydroxyl functional oil polyol acrylic graft copolymer.

In some embodiments of the invention, hydroxyl functional oil polyol acrylic graft copolymers are made into solvent and waterborne aqueous personal care compositions. Solvent-borne personal care compositions may contain, for non-limiting example, an ethylenically unsaturated monomer component that includes without limitation non-functional ethylenically unsaturated monomers such as, for non-limiting example, butyl acrylate, butyl methacrylate, methyl methacrylate, styrene, and the like, and optionally with lesser amounts of functional monomers such as, acid-acid monomers, hydroxy containing monomers and cationic monomers.

In some embodiments of the invention, hydroxyl functional monomers are added at a level of about 0 to about 30%, preferably about 0.1 to about 25% and more preferably about 1 to about 15% by weight of the ethylenically unsaturated monomer component mixture, and acid functional monomers are added at a level of about 0.1 to about 30%, preferably about 1 to about 50% and more preferably about 1 to about 25% by weight of the ethylenically unsaturated monomer component mixture. The water borne aqueous personal care compositions in some embodiments of the invention contain non-functional and hydroxyl functional monomers as listed above, with higher levels of acid functional monomer to render the composition water dispersible. In some embodiments, about 10 to about 50% by weight of the ethylenically unsaturated monomer component mixture is an acid functional monomer. In some embodiments, the acid functional monomer is methacrylic acid. The hydroxyl functional oil polyol acrylic graft copolymer is inverted into water by adding a neutralizing base, such as without limitation, ammonia or a tertiary amine such as without limitation, dimethyl ethanol amine, 2-Amino-2-methyl-1-propanol or triethanolamine. Final NV (non-volatile content by weight) is about 15 to about 40% by weight.

The epoxidized vegetable oil can be used alone or in combination with other epoxidized vegetable oils. Epoxidized vegetable oils can be prepared from vegetable oils by, for non-limiting example, adding hydrogen peroxide and formic or acetic acid to the vegetable oil, and then holding the mixture at an elevated temperature until some or all of the carbon-carbon double bonds are converted to epoxide groups.

Vegetable oils primarily include glycerides which are triesters of glycerol and fatty acids with varying degrees of unsaturation. For non-limiting example, epoxidized vegetable oils suitable for use in the invention can be made from vegetable oils (fatty acid triglycerides) such as, without limitation, esters of glycerol and fatty acids having an alkyl chain of about 12 to about 24 carbon atoms. Fatty acid glycerides, which are triglycerides in unsaturated glyceride oils, are generally referred to as drying oils or semidrying oils. Drying oils include, for non-limiting example, linseed oil, perilla oil and combinations thereof, while semidrying oils include, without limitation, tall oil, soy bean oil, safflower oil and combinations thereof. Triglyceride oils, in some embodiments, have identical fatty acid chains or alternatively have different fatty acid chains attached to the same glycerol molecule. In some embodiments, the oils have fatty acid chains containing non-conjugated double bonds. In some embodiments, single double bond or conjugated double bond fatty acid chains are used in minor amounts. Double bond unsaturation in glycerides can be measured by iodine value (number) which indicates the degree of double bond unsaturation in the fatty acid chains. Unsaturated fatty acid glyceride oils employed in some embodiments of the invention have an iodine value greater than about 25, from about 25 to about 210 and in another embodiment from about 100 to about 210.

Naturally occurring vegetable oils for use in the invention can be for non-limiting example, mixtures of fatty acid chains present as glycerides, and include, without limitation, a distribution of fatty acid esters of glyceride, where the fatty acid distribution may be random but within an established range that may vary moderately depending on the growing conditions of the vegetable source. Soy bean oil is employed in some embodiments wherein said soy bean oil comprises approximately about 11% palmitic, about 4% stearic, about 25% oleic, about 51% linolenic, and about 9% linoleic fatty acids, where oleic, linoleic and linolenic are unsaturated fatty acids. Unsaturated vegetable oils employed in some embodiments of the invention, include without limitation, glyceride oils containing non-conjugated unsaturated fatty acid glyceride esters such as, without limitation, linoleic and linolenic fatty acids.

Unsaturated glyceride oils include, without limitation, corn oil, cottonseed oil, grapeseed oil, hempseed oil, linseed oil, wild mustard oil, peanut oil, perilla oil, poppyseed oil, rapeseed oil, safflower oil, sesame oil, soy bean oil, sunflower oil, canola oil, tall oil, and mixtures thereof. Fatty acid glycerides for use in the invention include, for non-limiting example, those which contain linoleic and linolenic fatty acid chains, oils such as without limitation, hempseed oil, linseed oil, perilla oil, poppyseed oil, safflower oil, soy bean oil, sunflower oil, canola oil, tall oil, grapeseed oil, rattonseed oil, corn oil, and similar oils which contain high levels of linoleic and linolenic fatty acid glyceride. Glycerides can contain lesser amounts of saturated fatty acids in some embodiments. For non-limiting example, soy bean oil can be employed which contains predominantly linoleic and linolenic fatty acid glycerides. Combinations of such oils are employed in some embodiments of the invention. Vegetable oils can by fully or partially epoxidized by known processes, such as for non-limiting example, using acids such as, without limitation, peroxy acid for epoxidation of unsaturated double bonds of the unsaturated vegetable oil. Unsaturated glyceride oils employed in some embodiments include mono-, di-glycerides and mixtures thereof with tri-glycerides or fatty acid esters of saturated and unsaturated fatty acids. In some embodiments, the epoxidized vegetable oil comprises corn oil, cottonseed oil, grapeseed oil, hempseed oil, linseed oil, wild mustard oil, peanut oil, perilla oil, poppyseed oil, rapeseed oil, safflower oil, sesame oil, soy bean oil, sunflower oil, canola oil, tall oil, a fatty acid ester, monoglyceride or diglyceride of such oils, or a mixture thereof.

Commercially available sources of epoxidized vegetable oils suitable for use in the invention include, but are not limited to, epoxidized soy oil sold under the trade designations “VIKOLOX” and “VIKOFLEX 7170” available from Arkema, Inc, “DRAPEX 6.8” available from Chemtura Corporation, and “PLAS-CHECK 775” available from Ferro Corp. Other epoxidized vegetable oils for use in the invention include, for non-limiting example, epoxidized linseed oil sold under the trade designations “VIKOFLEX 7190” available from Arkema, Inc. and “DRAPEX 10.4” available from Chemtura Corporation, epoxidized cotton seed oil, epoxidized carthamus oil and mixtures thereof. Epoxidized soy bean oil is employed in some embodiments.

In some embodiments of the invention, the hydroxyl functional material includes, without limitation, propylene glycol, ethylene glycol, 1,3-propane diol, neopentyl glycol, trimethylol propane, diethylene glycol, a polyether glycol, a polyester, a polycarbonate, a polyolefin, a hydroxyl functional polyolefin, and mixtures thereof. The hydroxyl functional material includes an alcohol in some embodiments such as, without limitation, n-butanol, 2-ethyl hexanol, benzyl alcohol, and the like, alone, or in combination with diols or polyols.

In some embodiments, the hydroxyl functional material is present in an amount from about 1:99 to about 99:1, preferably from about 75:25 to about 99:1 in a weight ratio of hydroxyl functional material to epoxidized vegetable oil.

The acid catalyst employed to facilitate the reaction of the epoxidized vegetable oil with the hydroxyl functional material can be a strong acid catalyst such as, for non-limiting example, one or more sulfonic acids or another strong acid (an acid with a pKa about 3 or less), a triflic acid, a triflate salt of a metal of Group HA, HB, III A, IIIB or VIIIA of the Periodic Table of Elements (according to the IUPAC 1970 convention), a mixture of said triflate salts, or a combination thereof. In some embodiments, the amount of the acid catalyst can range from about 1 ppm to about 10,000 ppm, and alternatively from about 10 ppm to about 1,000 ppm, based on the total weight of the reaction mixture. Catalysts include, but are not limited to, the Group HA metal triflate catalysts, such as magnesium triflate, the Group HB metal triflate catalysts, such as zinc and cadmium triflate, the Group IIIA metal triflate catalysts, such as lanthanum triflate, the Group IIIB metal triflate catalysts, such as aluminum triflate, and the Group VIIIA metal triflate catalysts, such as cobalt triflate, and combinations thereof. The amount of the metal triflate catalyst can range, for non-limiting example, from about 10 to about 1,000 ppm, alternatively from about 10 to about 200 ppm, based on the total weight of the reaction mixture. Some embodiments of the invention employ a metal triflate catalyst in the form of a solution in an organic solvent. Examples of solvents include, without limitation, water, alcohols, such as n-butanol, ethanol, propanol, and the like, as well as aromatic hydrocarbon solvents, cycloaliphatic polar solvents such as, for non-limiting example, cycloaliphatic ketones (e.g. cyclohexanone), polar aliphatic solvents, such as, for non-limiting example, alkoxyalkanols, 2-methoxyethanol, non hydroxyl functional solvents, and mixtures thereof.

In some embodiments, an ethylenically unsaturated monomer component and an initiator are reacted with the hydroxyl functional oil polyol to form a hydroxyl functional oil polyol acrylic graft copolymer. The ethylenically unsaturated monomer component and the initiator can be added after the hydroxyl functional oil polyol is cooled. In some embodiments, the ethylenically unsaturated monomer component and initiator are added over about 2 hours. In some embodiments, the reaction product of the hydroxyl functional oil polyol, ethylenically unsaturated monomer component and initiator is cooled after about a 1 hour hold to form the hydroxyl functional oil polyol acrylic graft copolymer. Although the term “acrylic” is used in describing the hydroxyl functional oil polyol acrylic graft copolymer, the word acrylic is used in its broadest sense to include all ethylenically unsaturated monomer components.

The ethylenically unsaturated monomer component can be composed of a single monomer or a mixture of monomers. The ethylenically unsaturated monomer component includes, without limitation, non-functional monomers, acid monomers, hydroxy containing monomers, and cationic monomers. Suitable non-functional monomers include, without limitation, acrylic monomers, allylic monomers, acrylamide monomers, vinyl esters including without limitation, vinyl acetate, vinyl propionate, vinyl butyrates, vinyl benzoates, vinyl isopropyl acetates, and similar vinyl esters, vinyl halides including without limitation, vinyl chloride, vinyl fluoride and vinylidene chloride, vinyl aromatic hydrocarbons including without limitation, styrene, methyl styrenes and similar lower alkyl styrenes, chlorostyrene, vinyl toluene, vinyl naphthalene, vinyl aliphatic hydrocarbon monomers including without limitation, alpha olefins such as for non-limiting example, ethylene, propylene, isobutylene, and cyclohexene, as well as conjugated dienes such as for non-limiting example, 1,3-butadiene, methyl-2-butadiene, 1,3-piperylene, 2,3 dimethyl butadiene, isoprene, cyclohexane, cyclopentadiene, and dicyclopentadiene. Vinyl alkyl ethers include without limitation, methyl vinyl ether, isopropyl vinyl ether, n-butyl vinyl ether, and isobutyl vinyl ether. Acrylic monomers include without limitation, monomers such as for non-limiting example, lower alkyl esters of acrylic or methacrylic acid having an alkyl ester portion containing between about 1 to about 10 carbon atoms, as well as aromatic derivatives of acrylic and methacrylic acid, methyl acrylate and methacrylate, ethyl acrylate and methacrylate, n-octylacrylate, acrylamide, t-octylacrylamide, butylacrylamide, methylacylamide, butyl acrylate and methacrylate, propyl acrylate and methacrylate, 2-ethyl hexyl acrylate and methacrylate, cyclohexyl acrylate and methacrylate, decyl acrylate and methacrylate, isodecylacrylate and methacrylate, benzyl acrylate and methacrylate.

Suitable acid monomers include, without limitation, acrylic acid, methacrylic acid, maleic acid, itoconic acid, crotonic acid and the like.

Suitable hydroxy containing monomers include, without limitation, hydroxypropylmethacrylate, hydroxyethylacrylate, various glycidyl ethers reacted with acrylic and methacrylic acids, hydroxyl alkyl acrylates and methacrylates such as without limitation, hydroxyethyl and hydroxy propyl acrylates and methacrylates.

Suitable cationic monomers include, without limitation, t-butylaminoethylmethacrylate, amino acrylates and methacrylates.

In some embodiments, the weight ratio of the ethylenically unsaturated monomer component to the hydroxyl functional oil polyol is from about 1:99 to about 99:1, alternatively from about 5:95 to about 95:5, and alternatively from about 30:70 to about 70:30.

Various initiators are employed alone or in combination in some embodiments of the invention. In some embodiments, initiators with high grafting efficiencies are employed. Suitable initiators include, without limitation, azo compounds such as for non-limiting example, 2,2′-azo-bis(isobutyronitrile), 2,2′-azo-bis(2,4-dimethylvaleronitrile), and 1-t-butyl-azocyanocyclohexane), hydroperoxides such as for non-limiting example, t-butyl hydroperoxide and cumene hydroperoxide, peroxides such as benzoyl peroxide, caprylyl peroxide, di-t-butyl peroxide, ethyl 3,3′-di(t-butylperoxy) butyrate, ethyl 3,3′-di(t-amylperoxy) butyrate, t-amylperoxy-2-ethyl hexanoate, l,l,3,3-tetramethylbutyl-peroxy-2-ethylhexanoate, and t-butylperoxy pivilate, peresters such as for non-limiting example, t-butyl peracetate, t-butyl perphthalate, and t-butyl perbenzoate, as well as percarbonates, such as for non-limiting example, di(l-cyano-l-methylethyl)peroxy dicarbonate, perphosphates, t-butyl peroctoate, and the like and mixtures thereof.

In some embodiments, the initiator is present in an amount from about 0.1 to about 15%, and alternatively from about 1 to about 5%, based on the weight of the monomer mixture.

The temperature chosen for grafting the ethylenically unsaturated monomer component may vary with the half life of the selected initiator in some embodiments on the invention. For non-limiting example, at 130° C. t-butyl peroxy benzoate has a half life of about 30 minutes and can be employed for grafting. Dibenzoyl peroxide has a 30 minute half life at 100° C., and 100° C. could be a temperature to graft the hydroxyl functional oil polyol with dibenzoyl peroxide in some embodiments of the invention. Broadly, depending on the half life of the initiator used, the reaction can be carried out from about 50 to about 200° C.

In some embodiments, one or more mixtures of an initiator with or without a solvent is added after formation of the hydroxyl functional oil polyol acrylic graft copolymer to reduce the free monomer content. The compositions of the initiator and solvent in these one or more mixtures can be the same as or different than the compositions of these components used to form the hydroxyl functional oil polyol acrylic graft copolymer.

For personal care formulations the hydroxyl functional oil polyol acrylic graft copolymer can be provided in a solvent or as an emulsion of the copolymer as an oil-in-water system. In an embodiment, the polymer is supplied to the personal care formulation in C₁-C₄ alcohols or polyols or butyl cellosolve (2-butoxy ethanol) or combinations thereof.

For personal care formulations hydroxyl functional vegetable oil polyol acrylic graft copolymers will be present in the formulation from about 0.1 to about 20% based on the total dry weight of the composition. In another embodiment the hydroxyl functional vegetable oil polyol acrylic graft copolymers will be present in the formulation from about 0.2 to about 10% of the total dry formulation. In yet another embodiment, the hydroxyl functional vegetable oil polyol acrylic graft copolymers will be present from about 0.5 to about 5% of the total dry composition weight.

When the hydroxyl functional oil polyol acrylic graft copolymer contains (meth)acrylate monomers, such as methyl acrylate, the hydroxyl functional oil polyol acrylic graft copolymer will form an emulsion when added to water or aqueous formulations. This emulsion will provide the water and/or high humidity resistance when the personal care formulation is dried to form a film.

Such formulations can be used on either the hair or the skin. In applications where the formulation pertains to the hair, some non-limiting properties are that would be desirable are curl retention, stiffness, anti-flaking, high humidity curl retention, where high humidity curl retention is about 40% or greater, preferably about 50% or greater and more preferably about 75% or greater after 2 hours, and soft feel. In an embodiment, the hair care formulations containing the hydroxyl functional oil polyol acrylic graft copolymer will be hair styling spray applications containing at least 55% VOC solvents.

Some non-limiting examples of hair care products that could be formulated with the hydroxyl functional oil polyol acrylic graft copolymer are hair styling formulations, such as a pump spray, aerosol spray, combing/hair cream or mousse.

The waterproofing effect of the hydroxyl functional oil polyol acrylic graft copolymer may also be used in skin care products to provide such functions as sweat resistance, water resistance (such as when swimming), rub-off resistance and enhanced film forming properties. Some examples of formulations that may contain the hydroxyl functional oil polyol acrylic graft copolymer include, but are not limited to, sunscreens, moisturizers, medicaments and insect repellents.

The cosmetic and personal care compositions contain suitable “cosmetic and personal care actives”. Suitable cosmetic and personal care active agents include, for example, sunscreen agents or actives, aesthetic enhancers, conditioning agents, anti-acne agents, antimicrobial agents, anti-inflammatory agents, analgesics, anti-erythemal agents, antiruritic agents, antiedemal agents, antipsoriatic agents, antifungal agents, skin protectants, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, astringents, cleansers, sensates, antibiotics, anesthetics, steroids, tissue healing substances, tissue regenerating substances, hydroxyalkyl urea, amino acids, peptides, minerals, ceramides, biohyaluronic acids, vitamins, skin lightening agents, self tanning agents, coenzyme Q10, niacinimide, capcasin, caffeine, and any combination of any of the foregoing.

Suitable sunscreen agents or actives useful in the present invention include any particulate sunscreen active that absorbs, scatters, or blocks ultraviolet (UV) radiation, such as UV-A and UV-B. Non-limiting examples of suitable particulate sunscreen agents include clays, agars, guars, nanoparticles, native and modified starches, modified cellulosics, zinc oxide, and titanium dioxide and any combination of the foregoing. Modified starches include, for example, DRY-FLO®PC lubricant (aluminum starch octenylsuccinate), DRY-FLO®AF lubricant (corn starch modified), DRY-FLO® ELITE LL lubricant (aluminum starch octenylsuccinate (and) lauryl lysine), DRY-FLO® ELITE BN lubricant (aluminum starch octenylsuccinate (and) boron nitride), all commercially available from National Starch and Chemical Company.

The sunscreen agents may include those that form a physical and/or chemical barrier between the UV radiation and the surface to which they are applied. Non-limiting examples of suitable sunscreen agents include ethylhexyl methoxycinnamate (octinoxate), ethylhexyl salicylate (octisalate), butylmethoxydibenzoylmethane, methoxydibenzoylmethane, avobenzone, benzophenone-3 (oxybenzone), octocrylene, aminobenzoic acid, cinoxate, dioxybenzone, homosalate, methyl anthranilate, octocrylene, octisalate, oxybenzone, padimate O, phenylbenzimidazole sulfonic acid, sulisobenzone, trolamine salicylate and any combination of any of the foregoing

The cosmetic and personal care compositions can optionally include one or more aesthetic enhancers (i.e., a material that imparts desirable tactile, visual, taste and/or olfactory properties to the surface to which the composition is applied) and can be either hydrophilic or hydrophobic. Non-limiting examples of commercial aesthetic enhancers together with their INCI names that are optionally suitable for use in the present invention include PURITY®21C starch (zea maize (corn) starch) and TAPIOCA PURE (tapioca starch), as well as combinations thereof, that are available from Akzo Nobel Surface Chemistry, of Chicago, Ill.

Suitable conditioning agents include, but are not limited to, cyclomethicone; petrolatum; dimethicone; dimethiconol; silicone, such as cyclopentasiloxane and diisostearoyl trimethylolpropane siloxy silicate; sodium hyaluronate; isopropyl palmitate; soybean oil; linoleic acid; PPG-12/saturated methylene diphenyldiisocyanate copolymer; urea; amodimethicone; trideceth-12; cetrimonium chloride; diphenyl dimethicone; propylene glycol; glycerin; hydroxyalkyl urea; tocopherol; quaternary amines; and any combination thereof.

The cosmetic and personal care compositions can optionally include one or more adjuvants, such as pH adjusters, emollients, humectants, conditioning agents, moisturizers, chelating agents, propellants, rheology modifiers and emulsifiers such as gelling agents, colorants, fragrances, odor masking agents, UV stabilizer, preservatives, and any combination of any of the foregoing. Examples of pH adjusters include, but are not limited to, aminomethyl propanol, aminomethylpropane diol, triethanolamine, triethylamine, citric acid, sodium hydroxide, acetic acid, potassium hydroxide, lactic acid, and any combination thereof.

The cosmetic and personal care compositions may also contain preservatives. Suitable preservatives include, but are not limited to, chlorophenesin, sorbic acid, disodium ethylenedinitrilotetraacetate, phenoxyethanol, methylparaben, ethylparaben, propylparaben, phytic acid, imidazolidinyl urea, sodium dehydroacetate, benzyl alcohol, methylchloroisothiazolinone, methylisothiazolinone, and any combination thereof. In an embodiment of the invention, the cosmetic and personal care composition generally contains from about 0.001% to about 20% by weight of preservatives, based on 100% weight of total composition. In another embodiment, the composition contains from about 0.1% to about 10% by weight of preservatives, based on 100% weight of total composition.

The cosmetic and personal care compositions may optionally contain thickeners or gelling agents. Examples of such gelling agents include, but are not limited to, synthetic polymers such as the acrylic-based Carbopol® series of thickeners available from B. F. Goodrich, Cleveland, Ohio and associative thickeners such as Aculyn™, available from Rohm & Haas, Philadelphia, Pa. Other exemplary gelling agents include, cellulosic thickeners, such as derivatized hydroxyethyl cellulose and methyl cellulose, starch-based thickeners, such as acetylated starch, and naturally occurring gums, such as agar, algin, gum arabic, guar gum and xanthan gum. Thickeners and rheology modifiers may also include without limitation acrylates/steareth-20 itaconate copolymer, acrylates/ceteth-20 itaconate copolymer, potato starch modified, hydroxypropyl starch phosphate, acrylates/aminoacrylates/C10-30 alkyl PEG-20 itaconate copolymer, carbomer, acrylates/C10-30 alkyl acrylate crosspolymer, hydroxypropylcellulose, hydroxyethylcellulose, sodium carboxymethylcellulose, polyacrylamide (and) C13-14 isoparaffin (and) laureth-7, acrylamides copolymer (and) mineral oil (and) C13-14 isoparaffin (and) polysorbate 85, hydroxyethylacrylate/sodium acrylol dimethyltaurate copolymer, and hydroxyethylacrylate/sodium acrylol dimethyltaurate copolymer.

In an embodiment of the invention, the cosmetic and personal care composition is a hair cosmetic composition. Optional conventional additives may also be incorporated into the hair cosmetic compositions of this invention to provide certain modifying properties to the composition. Included among these additives are silicones and silicone derivatives; humectants; moisturizers; plasticizers, such as glycerine, glycol and phthalate esters and ethers; emollients, lubricants and penetrants, such as lanolin compounds; fragrances and perfumes; UV absorbers; dyes, pigments and other colorants; anticorrosion agents; antioxidants; detackifying agents; combing aids and conditioning agents; antistatic agents; neutralizers; glossifiers; preservatives; proteins, protein derivatives and amino acids; vitamins; emulsifiers; surfactants; viscosity modifiers, thickeners and rheology modifiers; gelling agents; opacifiers; stabilizers; sequestering agents; chelating agents; pearling agents; aesthetic enhancers; fatty acids, fatty alcohols and triglycerides; botanical extracts; film formers; and clarifying agents. These additives are present in small, effective amounts to accomplish their function, and generally will comprise from about 0.01 to about 10% by weight, and from about 0.01 to about 20% by weight total, based on the weight of the composition.

The hair cosmetic composition may optionally be a mousse. For mousses, the solvent may be a lower (C₁₋₄) alcohol, particularly methanol, ethanol, propanol, isopropanol, or butanol, although any solvent known in the art may be used.

Optionally, an embodiment of the invention may also comprise a spray. For sprays propellants include any optional propellant(s). Such propellants include, without limitation, ethers, such as dimethyl ether; one or more lower boiling hydrocarbons such as C₃-C₆ straight and branched chain hydrocarbons, for example, propane, butane, and isobutane; halogenated hydrocarbons, such as, hydrofluorocarbons, for example, 1,1-difluoroethane and 1,1,1,2-tetrafluoroethane, present as a liquefied gas; and the compressed gases, for example, nitrogen, air and carbon dioxide.

In another aspect, the present invention relates to a method of preparing a personal care composition comprising mixing a vegetable oil polyol acrylic graft copolymer to at least one cosmetic or personal care active ingredient, wherein the graft copolymer is obtained from a modified epoxidized vegetable oil that has been polymerized in the presence of at least one ethylenically unsaturated monomer.

In yet another aspect, the present invention is directed to the use of a hydroxyl functional vegetable oil polyol acrylic graft copolymer in a personal care composition wherein the graft copolymer is obtained from a modified epoxidized vegetable oil polymerized in the presence of at least one ethylenically unsaturated monomer.

The invention will now be further described in connection with the following Examples which are not intended to limit the scope thereof. Unless otherwise stated, all parts and percentages refer to parts and percentages by weight.

### Experimental:

The table below is a list of all the ingredients used in the following experiments and formulations found in the experimental section. The chemical name is the INCI or common name and the trade name is the manufacturer or supplier designation.

TABLE 1

| Chemical Name | Trade name | Supplier |
|---|---|---|
| Acrylates, Butylaminoethyl Methacrylate | Amphomer ® LV-71 | AkzoNobel Surface Chemistry, |
| Copolymer, Octylacrylamide | | Global Personal Care |
| Acrylates Copolymer | Balance ® CR | AkzoNobel Surface Chemistry, |
| | | Global Personal Care |
| EDTA | Dissolvine ® 220-S | AkzoNobel Surface Chemistry, |
| | | Global Personal Care |
| Aluminum Starch Octenylsuccinate | DRY-FLO ® PC | AkzoNobel Surface Chemistry, |
| | starch | Global Personal Care |
| 2-Amino-2-methyl-1-propanol | AMP-95 | ANGUS Chemical Company |
| Acrylates/Hydroxyesters Acrylates | Acudyne ™ 180 | Dow Corning Corp |
| Copolymer | | |
| Carbomer | Carbopol ® 940 | Lubrizol Advanced Materials |
| Octocrylene | Neo Heliopan ® 303 | Symrise Inc |
| Homosalate | Neo Heliopan ® | Symrise Inc |
| | HMS | |
| Benzophenone-3 | Neo Heliopan ® BB | Symrise Inc |
| Avobenzone | Neo Heliopan ® 357 | Symrise Inc |
| Phenethyl Benzoate | FINSOLV SUN ® | Innospec Active Chemicals |
| Stearic acid | Emersol ® 132 | Chemical Associates, Inc. |
| Glyceryl Stearate | Cutina GMS-SE - | Cognis Corp., Care Chemicals |
| | Cognis | |
| Benzyl Alcohol | Microcare BNA | Thor Specialty Chemicals Inc |
| Dimethicone | Dow Corning 200, | Dow Corning Corp |
| | 100 cts | |
| Fumed Silica | Cab-O-Sil ® M-5 | Cabot Corp. |
| Alkylated Polyvinyl Pyrrolidone with | Ganex ® V-220 | INTERNATIONAL SPECIALTY |
| Eicosene 30/70 ratio | | PRODUCTS |
| Phenoxyethanol, Isopropylparaben, | Liquipar ® PE | INTERNATIONAL SPECIALTY |
| Isobutylparaben, Butylparaben. | | PRODUCTS |
| Acrylates/Hydroxyesters Acrylates | ACUDYNE ® 180 | DOW Chemical |
| Copolymer | | |

### High Humidity Curl Retention

The curl retention properties of polymeric hair spray resins are measured in an environmental chamber (Thermal Product Solutions—Tenney Humidity Chamber; model TH27) at 70° F./90% Relative Humidity over a period of desired length of time. The change in percentage curl retention versus time is determined to illustrate differences among formulations.

A day prior to testing, the curls are prepared in the following manner:
Wet 6″ swatch and comb through to remove snarls, squeeze out excess water.
Curl hair into a coil configuration along the length of the Teflon mandrel, making sure to keep the curl tight and continuous along the mandrel, with no gaps.
Secure hair on mandrel with plastic Tygon (pre-cut) clips, one on each end of the curl.
Place finished curls in 110° F. oven overnight.
   Testing of the formulation is carried out in the following manner:
Remove dried curls from 110° F. oven and allow to cool for 30 minutes at room temperature.
Carefully remove clips and curl from mandrel.
Suspend the curl from the bound end.
Apply uniformly four bursts (from a pump spray) of the desired formulation to the front and four bursts to the back of the curl (total of eight bursts) from a distance of 6″
A total of nine curls are sprayed for each formulation.
Lay the freshly sprayed curls on a clean tray (foil-covered) and allow to air dry at room temperature for one hour.
Re-Suspend the curl from the bound end
Take initial curl height reading.
Place the curls environmental chamber.
After boards are in chamber, record curl lengths at the 15 minute, 30 minute, 60 minute, and 90 minute, 2 hour, 3 hour, 4 hour, 5 hour, and 24 hour (if needed) intervals.

### Data Analysis:

Average for each formulation was determined, as well as the least square difference to determine the statistical significance of the data.

### In-Vitro Water Resistance SPF Testing

The sunscreen samples were tested using the IMS In-Vitro Water Resistance Protocol (In Vitro SPF/UVA measurements made both prior to, and after the samples have been immersed in a controlled temperature water bath: 40 C—agitated at 300 rpm—for 80 minutes). All sample tests were run and reported by:
IMS Inc.
110 Marginal Way
Portland, Me. 04101 USA
(203) 876-9400 ext. 218 Fax (203) 876-9445

### EXAMPLE 1

### Preparation of Hydroxyl Functional Soy Oil Polyol Acrylic Graft Copolymer

18.6 grams of propylene glycol, 81.4 grams of epoxidized soy bean oil (available from Ferro Corp, 4150 E 56th St, Cleveland, Ohio 44105-4800) and 0.021 grams of Nacure A-218 (available from King Industries, Inc, Science Road, Norwalk, Conn. 06852) were stirred under nitrogen and warmed to 150 C. The initial exotherm was controlled at <160 C, and the mixture was held at 150 C for about 2 hours. Oxirane titration at this point indicated >99.9% conversion of the epoxide groups. 100 g of butyl cellusolve was added and the mixture was cooled to 100 C under nitrogen. A mixture of 24.2 grams of methacrylic acid, 20.8 grams of methyl methacrylate, 55.0 grams of n-butyl methacrylate and 2.0 grams of dibenzoyl peroxide were then had into the reaction mixture over 2 hours. The resultant mixture was held for 1 hour at 100 C and 59 grams of butyl cellusolve was added and the resultant mixture was cooled, affording a hydroxyl functional oil polyol acrylic graft copolymer.

The resulting polymer was than neutralized with dimethyl ethanol amine using the following procedure:

45 grams of the above polymer was heated to about 100° C. under agitation. 15 grams of a 33% aqueous solution of dimethyl ethanol amine was slowly added over a period of one minute. The mixture was further mixed for 10 minutes. 40 grams water was added over about 5 minutes to give about 25% polymer solids. The temperature was allowed fall with the addition of water to about 80° C. The resulting emulsion was stirred for about 1 hour at 80° C. It was than cooled to room temperature and poured out. The resulting pH of the emulsion was about 8.

### EXAMPLE 2

### Preparation of 55% VOC Pump Hair Spray Formulation and Quantitative Performance Data

A sample 55% VOC hair spray formulation was prepared including the components as listed in Table 2.

Using the polymer in Example 1, the formulation was prepared in the following manner: Ethanol was added to water and mixed thoroughly until homogeneous. Mixing was carried out in a beaker using a magnetic stirrer. The polymer in Example 1 was than added and mixed thoroughly until homogeneous.

For Amphomer® LV-71 the formulation was prepared in the following manner: Ethanol was added to water and mixed thoroughly until homogeneous. Mixing was carried in a beaker using a magnetic stirrer. A desired amount of AMP-95 was added and mixed until homogeneous. Finally Amphomer® LV-71 was added and mixed until fully dissolved.

TABLE 2
Basic 55% VOC pump hair spray formulation

| Component | Actual wt, gms | |
|---|---|---|
| Soy Polymer neutralized with dimethyl | 40 | — |
| ethanol amine (Polymer of Example 1) | | |
| Amphomer ® LV71 neutralized | — | 10.05 |
| AMP-95 | — | 2.02 |
| Ethanol | 110 | 110.06 |
| Water | 50 | 77.9 |

The 55% VOC hair spray formulation was tested to determine the percent High Humidity Curl retention as compared to a conventional synthetic polymer, Amphomer® LV-71. The results are shown in Table 3.

TABLE 3
Pump hair spray HHCR data (High Humidity Curl Retention)

% hold

| | Amphomer ® | Polymer of |
|---|---|---|
| Time, hrs | LV-71 | Example 1 |
| 0 | 100 | 100 |
| 0.25 | 86 | 88 |
| 0.5 | 70 | 76 |
| 1 | 58 | 60 |
| 1.5 | 45 | 53 |
| 2 | 39 | 46 |
| 3 | 36 | 42 |
| 4 | 34 | 39 |
| 5 | 32 | 35 |

The results in Table 3 indicate that the data show that the inventive hydroxyl functional soy oil polyol acrylic graft copolymer (Polymer of Example 1) provides better percent HHCR than the synthetic polymer Amphomer® LV-71.

### EXAMPLE 3

### Qualitative (Subjective) Performance Data

A paired comparison was conducted to compare the inventive Sample Polymer 1 compared to a conventional polymer in a hairspray formulation. Significance was reported at the 95% confidence level. The data is reported indicates whether the inventive functional oil polyol acrylic graft copolymer performed better (+), equal to (=) or inferior (−) to the conventional synthetic polymer. The polymers were compared based on:

### Subjective Properties Description:

### Beading:

Visually examine the swatch for dried polymer beads. Choose the swatch which has more beading.

### Gloss:

Gently handle the swatches so as not to break the films. Visually inspect the swatches to determine which has more shine/gloss.

### Stiffness:

Gently handle swatches and feel for differences in stiffness. Using two fingers, hold the middle of the swatch in a horizontal position—does one bend more than the other? Choose the one that is more rigid.

### Spring:

While holding the swatch in one hand, gently pull on an edge with the other hand three times only. Look for spring back, and bounce. The more elastic the better the Spring.

### Webbing:

While holding the swatch in both hands, gently pull outward on the edges approx. 4″. (Do this three times only to avoid damage to the bonds. If the bonds are destroyed then the dry combing may appear to be easier to comb). The more net like the better the Webbing.

### Dry Comb:

Comb through each swatch (5) times and evaluate ease of combing. Choose the one that combs more easily.

### Flake:

Visually inspect both swatches after combing. Check the teeth of the comb for flake accumulation. Holding the swatch at the bound end run your fingernail down the length of the tress then inspect. Choose the one with more flakes.

### Anti-Static:

Holding swatch at bound end comb through vigorously 10 times then evaluate for extent of fly aways generated. Choose the one with more fly aways.

### Feel:

Handle swatches and determine preference. Choose the one that feels more soft and silky
The results of the subjective tests are as follows:

TABLE 4
Polymer of Example 1 vs. Amphomer LV-71 in 55% VOC Pump Hairspray

| Sample | Beading | Gloss | Stiffness | Spring | Webbing | Dry Comb | Flake | Static | Feel |
|---|---|---|---|---|---|---|---|---|---|
| Soy | = | = | = | = | − | = | = | = | = |

### EXAMPLE 4

### Additional Solvent Testing

A common solvent used in hair spray is ethanol. To test applicability of the polymer of Example 1 in different solvent, such as isopropyl alcohol, additional testing was conducted. The formulations are shown in table 5. Subjective evaluation of the performance of the polymer in example 1 was compared against Acrylates/Hydroxyesters Acrylates Copolymer (ACUDYNE® 180) manufactured by Dow Chemical. ACUDYNE® 180 is a known hair fixative and styling polymer used in hair spray applications.

TABLE 5
Other Formulation tested (MIR compliant)

| | Formula #1 | | Formula # 2 | |
|---|---|---|---|---|
| | (Isopropanol-Free) | | (With Isopropanol) | |
| Component | Wt, gms | | Wt, gms | |
| Soy Polymer neutralized | 40 | | 40 | |
| with dimethyl ethanol | | | | |
| amine (Polymer Examle 1) | | | | |
| ACUDYNE ® 180 | — | 20.6 | — | 20.6 |
| AMP-95 | — | 1.95 | — | 1.95 |
| Ethanol | 90 | 90 | 66 | 66 |
| Ispopropanol | — | — | 60 | 60 |
| DI Water | 70 | 89 | 34 | 53 |

* Note:
ACUDYNE ® 180 was neutralized with AMP-95

Subjective results are shown in Table 6. The results shows that for most categories of property, such as beading, gloss etc the polymer of the invention in example 1 performs in similar manner as ACUDYNE® 180

TABLE 6

| | | Beading | Gloss | Stiffness | Spring | Webbing | Dry Comb | Flake | Static | Feel |
|---|---|---|---|---|---|---|---|---|---|---|
| Formula #1 | Soy (EXP 99201) | = | = | = | = | = | = | = | = | = |
| (Isopropanol- | Sample Polymer 1 | | | | | | | | | |
| Free) | ACUDYNE ® 180 | | | | | | | | | |
| | Soy (EXP 99201) | = | = | − | − | = | + | + | = | = |
| | Sample Polymer 1 | | | | | | | | | |
| Formula #2 | Soy (EXP 99201) | = | = | = | = | = | = | = | − | = |
| (with | Sample Polymer 1 | | | | | | | | | |
| Isopropanol) | ACUDYNE ® 180 | | | | | | | | | |
| | Soy (EXP 99201) | = | = | − | − | = | = | = | = | + |
| | Sample Polymer 1 | | | | | | | | | |

### EXAMPLE 5

### Emulsion Sunscreen

Emulsion sunscreen formulation with a target SPF of 50 was prepared using the polymer of Example 1. In vitro SPF testing was carried out on pre- and post-immersion in water to test for film forming and water resistance capability of the formulation. The data was compared with an emulsion sunscreen formulation containing a commercial product, Ganex 220A, available from International Specialty Products of Wayne, N.J. All testing was carried out by an external lab using their proprietary protocol. The following parameters were used:

Temperature of water bath—40° C.

Agitation rate—300 rpm

Immersion time: 80 minutes

The sunscreen formulations were prepared in following manner using various ingredient and amounts as listed in table 7:

Phase A—Dissolvine 220-S was added to water and heated to ˜75° C., to that Carbomer was added under vigorous mixing. After obtaining a uniform mix, Polymer of Example 1 was added followed by the neutralizer (TEA). In a separate container, Phase C was mixed and heated to ˜75° C. Phase C was then added to Phase A/B under vigorous mixing to obtain a uniform emulsion. The emulsion was cooled to ˜50° C. and Phase D was added. The product was than cooled to room temp under slow/moderate agitation. In case of Ganex V220, it was added to the oil phase (Phase C) instead of the water phase. If needed the pH of the samples were adjusted to 8 +/−0.25, using TEA.

TABLE 7
Sunscreen Formulation

| | | Percents |
|---|---|---|
| Phase A - water phase | | |
| | Water (Aqua) | Balance |
| | Dissolvine 220-S (EDTA) | 0.05 |
| | Carbopol 940 (Carbomer) | 0.25 |
| | Polymer of Example 1 | 2.00 |
| Phase B - Neutralizer | | |
| | Triethanolamine | 1.25 |
| Phase C - Oil phase | | |
| | Octocrylene | 10.00 |
| | Homosalate | 3.00 |
| | Neoheliopan BB | 6.00 |
| | Neoheliopan 357 | 3.00 |
| | Phenethyl Benzoate | 5.00 |
| | Stearic Acid | 2.00 |
| | Glyceryl Stearate SE | 2.00 |
| | Benzyl Alcohol | 1.00 |
| | Dimethicone | 0.50 |
| | Silica | 0.50 |
| | Ganex V220 | 2.00 |
| Phase D | | 6.00 |
| | Liquapar PE | 1.00 |
| | Dry Flo PC | 5.00 |

In-vitro SPF and water resistance results for emulsion sunscreen are shown below:

TABLE 8

| | VP/Eicosene Copolymer | | Polymer Exampe 1 | |
|---|---|---|---|---|
| | Pre- | Post- | Pre- | Post- |
| | immersion | immersion | immersion | immersion |
| SPF | 51 | 28 | 56 | 72 |
| UVA/UBV | 0.96 | 0.82 | 1.01 | 1.00 |
| ratio | | | | |

The data shows that the SPF rating to the formulation contain Polymer of Example 1 remains above 50 after immersion in water for 80 minutes, thus showing a significant improvement in water resistance capability. On the other hand, the data for a comparative sunscreen formulation containing Ganex 220A shows the SPF rating of the formulation drops below 50 after immersion. Thus, the inventive sunscreen formulation shows improved water resistance capability compared to a typical conventional sunscreen formulation.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described herein, the invention is not intended to be limited to the details shown. Rather, various modifications may be made in the details within the range and scope of equivalents of the claims and without departing from the spirit and scope of the invention.

What is claimed:

1. A personal care composition comprising:
a hydroxyl functional vegetable oil polyol acrylic graft copolymer obtained from a modified epoxidized vegetable oil polymerized in the presence of at least one ethylenically unsaturated monomer: and at least one cosmetic or personal care active ingredient.

2. The personal care composition of claim 1 wherein the modified epoxidized vegetable oil is derived from an ester of a glycerol and fatty acid having an alkyl chain of 12 to 24 carbon atoms.

3. The personal care composition of claim 1 wherein the modified epoxidized vegetable oil is derived from an unsaturated glyceride oil.

4. The personal care composition of claim 3 wherein the modified epoxidized vegetable oil is derived from an unsaturated glyceride oil selected from the group consisting of corn oil, cottonseed oil, grapeseed oil, hempseed oil, linseed oil, wild mustard oil, peanut oil, perilla oil, poppyseed oil, rapeseed oil, safflower oil, sesame oil, soy bean oil, sunflower oil, canola oil, tall oil, and mixtures thereof.

5. The personal care composition of claims 1 to 4 wherein the at least one ethylenically unsaturated monomer is selected from the group consisting of at least one non-functional monomer, at least one acid functional monomer, at least one hydroxyl monomer, at least one cationic monomer and combinations thereof.

6. The personal care composition of claims 1 to 5 wherein the at least one non-functional monomer is selected from the group consisting of acrylic monomers, allylic monomers, acrylamide monomers, vinyl esters, vinyl halides, vinyl aromatic hydrocarbons, vinyl toluene, vinyl naphthalene, vinyl aliphatic hydrocarbon monomers, vinyl alkyl ethers, conjugated dienes and combinations thereof.

7. The personal care composition of claim 6 wherein the at least one non-functional monomer is selected from the group consisting of butyl methacrylate, methyl methacrylate and styrene and mixtures thereof.

8. The personal care composition of claims 1 to 5 wherein the at least one acid functional monomer is selected from the group consisting of acrylic acid, methacrylic acid, maleic acid, itoconic acid, crotonic acid and combinations thereof.

9. The personal care composition of claims 1 to 5 wherein the at least one hydroxyl containing monomer is selected from the group consisting of hydroxpropylmethacrylate, hydroxyethylacrylate glycidyl ether reacted with acrylic and methacrylic acids, hydroxyl alkyl acrylates, methacrylates, and combinations thereof.

10. The personal care composition of claims 1 to 5 wherein the at least one cationic monomer is selected from the group consisting of t-butylaminoethylmethacrylate, amino acrylates, methacrylates and combinations thereof.

11. The personal care composition of claims 1 to 5 wherein the at least one ethylenically unsaturated monomer is a mixture of a non-functional monomer and an acid functional monomer.

12. The personal care composition of claim 1 wherein the personal care composition is a solvent-borne personal care composition.

13. The personal care composition of claim 1 wherein the personal care composition is a water-borne personal care composition.

14. The personal care composition according to any one of the preceding claims wherein the hydroxyl functional vegetable oil polyol acrylic graft copolymer is present in the composition from 0.1 to 10 percent of the total composition.

15. The personal care composition of any of the preceding claims wherein the personal care composition is selected from the group consisting of a pump hair spray, an aerosol hair spray, a combing/hair cream, a mousse, a sunscreen, a moisturizer, a medicament, an insect repellent and combinations thereof.

16. The personal care composition of claim 14 wherein the hair care composition further comprises silicone or a silicone derivative or a C₁₋₄ alcohol as a solvent or combinations thereof.

17. The personal care composition of any one of the preceding claims wherein the at least one cosmetic or personal care active ingredient is selected from the group consisting of sunscreen agents or actives, aesthetic enhancers, conditioning agents, anti-acne agents, antimicrobial agents, anti-inflammatory agents, analgesics, anti-erythemal agents, antiruritic agents, antiedemal agents, antipsoriatic agents, antifungal agents, skin protectants, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, astringents, cleansers, sensates, antibiotics, anesthetics, steroids, tissue healing substances, tissue regenerating substances, hydroxyalkyl urea, amino acids, peptides, minerals, ceramides, biohyaluronic acids, vitamins, skin lightening agents, self tanning agents, coenzyme Q10, niacinimide, capcasin, caffeine and combinations thereof.

18. A method of preparing a personal care composition comprising mixing a vegetable oil polyol acrylic graft copolymer to at least one cosmetic or personal care active ingredient, wherein the graft copolymer is obtained from a modified epoxidized vegetable oil that has been polymerized in the presence of at least one ethylenically unsaturated monomer.

19. Use of a hydroxyl functional vegetable oil polyol acrylic graft copolymer in a personal care composition wherein the graft copolymer is obtained from a modified epoxidized vegetable oil polymerized in the presence of at least one ethylenically unsaturated monomer.
